(19) United States
(12) Patent Application Publication
(10) Pub. No.: US 2005/0019798 A1
(43) Pub. Date: Jan. 27, 2005

(54) Methods to predict death from breast cancer

(76) Inventor: Michael Kattan, Cleveland Heights, OH (US)

Correspondence Address: Schwegman, Lundberg, Woessner & Kluth, P.A., P.O. Box 2938, Minneapolis, MN, 55402, US

(21) Appl. No.: 10/857,445

(22) Filed: May 28, 2004

Related U.S. Application Data
(60) Provisional application No. 60/474,644, filed on May 30, 2003.

(51) Int. Cl. C12Q 1/68; G01N 33/574; G06F 19/00; G01N 33/48; G01N 33/50

(52) U.S. Cl. 435/6; 435/7.23; 702/19

(57) ABSTRACT

A method and system to predict disease-specific death in patients with breast cancer.

### CROSS-REFERENCE TO RELATED APPLICATIONS

The present application claims the benefit under 35 U.S.C. § 119(e) of the filing date of U.S. Application Ser. No. 60/474,644, filed May 30, 2003, the disclosure of which is incorporated by reference herein.

### BACKGROUND OF THE INVENTION

The complexity of the decision regarding adjuvant therapy for women with breast cancer is widely documented. It is clear that the benefits of adjuvant therapy are modest, and that these must be balanced against its toxicities (Simes et al., 2001). Essentially, four issues drive the adjuvant therapy decision: the risk of relapse without the therapy, the toxicity of the therapy, its efficacy, and patient preferences (Mincey et al., 2002). It has been recognized that improving the ability to predict the first piece of information, i.e., the risk of relapse in the absence of adjuvant therapy, should improve decision making regarding adjuvant therapy (Hillner et al., 1992).

The Nottingham Prognostic Index (NPI), an established and validated model for predicting disease-specific survival for women with breast cancer, assumes that the woman does not receive adjuvant therapy. It is a simple, easy-to-use equation that depends on tumor size, grade, and lymph node score (D'Eredita et al., 2001), and it places a woman into one of three risk groups. However, a continuous prediction model has been shown in other cancers to provide greater predictive accuracy than the placement of patients into risk groups (Kattan et al., 2002; Kartan et al., 2000).

What is needed is an improved method to predict outcome for breast cancer patients.

### SUMMARY OF THE INVENTION

The invention provides methods, apparatus and nomograms to predict disease-specific death of a breast cancer patient in the absence of adjuvant therapy. In one embodiment, the invention includes correlating the value or score from clinical and/or pathological data, for example, in a nomogram, to predict patient outcome. For instance, the methods, apparatus or nomograms may be employed after surgery for breast cancer, e.g., including mastectomy and/or auxiliary lymph node dissection, but prior to adjuvant therapy for breast cancer to predict the risk of disease-specific death of the patient in the absence of adjuvant therapy.

As described herein, a total of 519 women were treated by mastectomy and auxiliary lymph node dissection who met the following inclusion criteria: confirmation of invasive mammary carcinoma, no neoadjuvant or adjuvant systemic therapy, no previous history of cancer, and node negative by routine histopathologic analysis. Data from these patients and competing risk analyses were used to develop a model to predict death more accurately by using a continuous prediction model. Enhanced pathologic analysis was performed on the auxiliary lymph node tissue blocks by sectioning and staining with hematoxylin and eosin (H&E) and immuno-histochemistry (IHC). Accuracy was measured using the concordance index and jackknife predictions from the model were compared with NPI predictions. The probability of death from breast cancer within 15 years was 20%. A model was constructed using age, multifocality, tumor size, tumor grade, lymphovascular invasion, and enhanced staining. Based on competing risks regression analysis, disease-specific death in the model was predicted more accurately than with the NPI.

Thus, as described herein, various factors in humans are prognostically useful, and may optionally be employed in conjunction with other markers for neoplastic disease such as those for breast cancer, e.g., in a nomogram to predict outcome in patients. In one embodiment, the prognosis is based on a computer derived analysis of data of the amount, level or other value (score) for one or more, e.g., two, three, four or more, of those factors. Data may be input manually or obtained automatically from an apparatus for measuring the amount, level or value of one or more factors.

Accordingly, the invention provides a method and apparatus, e.g., a computerized tool, to predict disease specific death with improved accuracy which is useful for counseling breast cancer patients on their need for adjuvant therapy. In one embodiment, the invention provides a method to determine the risk of disease-specific death of a patient after mastectomy and/or auxiliary lymph node dissection for breast cancer. The method comprises detecting or determining a score for one or more patient factors and then correlating those scores with the risk of disease-specific death of the patient in the absence of adjuvant therapy.

In one embodiment, the invention provides a method to determine the risk of disease-specific death in a breast cancer patient. The method includes detecting or determining one or more factors including tumor size, tumor grade, lymphovascular invasion and/or tumor tissue staining, in a breast cancer patient prior to adjuvant therapy. The values or scores for the one or more of the factors are correlated to the risk of disease-specific death of the patient in the absence of adjuvant therapy.

Further provided is a method to determine the prognosis of a breast cancer patient in the absence of adjuvant therapy. The method includes inputting test information to a data input means, wherein the information includes scores or values for one or more factors including tumor size, tumor grade, lymphovascular invasion and/or tumor tissue staining in a breast cancer patient prior to adjuvant therapy. A software for analysis of the test information is executed and the test information analyzed so as to provide the prognosis of the patient in the absence of adjuvant therapy.

Also provided is a method for predicting a probability of disease-specific death in a breast cancer patient. The method includes correlating one or more factors for the patient to a functional representation of one or more factors determined for each of a plurality of persons previously diagnosed with breast cancer and not having been treated with adjuvant therapy, so as to yield a value for total points for the patient. The factors for each of a plurality of persons is correlated with the probability of disease-specific death for each person in the plurality of persons. The one or more factors include tumor size, tumor grade, lymphovascular invasion and/or tumor tissue staining. The functional representation includes a scale for one or more of tumor size, tumor grade, lymphovascular invasion and/or tumor tissue staining, a total points scale, and a predictor scale, wherein the scales for tumor size, tumor grade, lymphovascular invasion and/or tumor tissue staining each have values on the scales which can be correlated with values on the points scale, and wherein the total points scale has values which may be correlated with values on the predictor scale. The value on the total points scale for the patient is correlated with a value on the predictor scale to predict the probability of disease-specific death in the patient in the absence of adjuvant therapy.

In addition, the invention provides an apparatus. The apparatus includes a data input means, for input of test information comprising one or more of tumor size, tumor grade, lymphovascular invasion and/or tumor tissue staining in a breast cancer patient prior to adjuvant therapy; a processor, executing a software for analysis of tumor size, tumor grade, lymphovascular invasion and/or tumor tissue staining; wherein the software analyzes the tumor size, tumor grade, lymphovascular invasion and/or tumor tissue staining and provides the risk of disease-specific death in the patient in the absence of adjuvant therapy.

An apparatus for predicting a probability of disease-specific death in a breast cancer patient in the absence of adjuvant therapy is further provided. The apparatus includes: a correlation of one or more factors for each of a plurality of persons previously diagnosed with breast cancer and not having been treated with adjuvant therapy with the incidence of disease-specific death for each person of the plurality of persons, wherein the one or more factors include tumor size, tumor grade, lymphovascular invasion and/or tumor tissue staining; and a means for comparing an identical set of factors determined from a patient having breast cancer to the correlation to predict the quantitative probability of disease-specific death in the patient in the absence of adjuvant therapy.

In one embodiment, a nomogram for the graphic representation of a quantitative probability of disease-specific death in a patient with breast cancer is provided. The nomogram includes a plurality of scales and a solid support, the plurality of scales being disposed on the support and comprising a scale of one or more of tumor size, tumor grade, lymphovascular invasion and/or tumor tissue staining, a points scale, a total points scale and a predictor scale, wherein the one or more scales for tumor size, tumor grade, lymphovascular invasion and/or tumor tissue staining each has values on the scales, and wherein the scales for tumor size, tumor grade, lymphovascular invasion and/or tumor tissue staining are disposed on the solid support with respect to the points scale so that each of the values on tumor size, tumor grade, lymphovascular invasion and/or tumor tissue staining, can be correlated with values on the points scale, wherein the total points scale has values on the total points scale, and wherein the total points scale is disposed on the solid support with respect to the predictor scale so that the values on the total points scale may be correlated with values on the predictor scale, such that the values on the points scale correlating with the tumor size, tumor grade, lymphovascular invasion and/or tumor tissue staining of the patient can be added together to yield a total points value, and the total points value can be correlated with the predictor scale to predict the quantitative probability of disease-specific death.

In another embodiment, the invention provides a method to predict a pre-adjuvant therapy prognosis in a breast cancer patient. The method includes determining a one or more factors for a patient, which one or more factors includes tumor size, tumor grade, lymphovascular invasion and/or tumor tissue staining, matching the factors to the values on the scales of a nomogram of the invention; determining a separate point value for each of the factors; adding the separate point values together to yield a total points value; and correlating the total points value with a value on the predictor scale of the nomogram to determine the prognosis of the patient in the absence of therapy.

Also provided is an apparatus for predicting a probability of disease-specific death in a patient with breast cancer. The apparatus includes: a scale for one or more of tumor size, tumor grade, lymphovascular invasion and/or tumor tissue staining, a points scale, a total points scale and a predictor scale, wherein the scales for tumor size, tumor grade, lymphovascular invasion and/or tumor tissue staining each has values on the scales, and wherein the scales for tumor size, tumor grade, lymphovascular invasion and/or tumor tissue staining are disposed so that each of the values for the tumor size, tumor grade, lymphovascular invasion and/or tumor tissue staining can be correlated with values on the points scale, wherein the total points scale has values on the total points scale, and wherein the total points scale is disposed on the solid support with respect to the predictor scale so that the values on the total points scale may be correlated with values on the predictor scale, such that the values on the points scale correlating with the tumor size, tumor grade, lymphovascular invasion and/or tumor tissue staining of the patient can be added together to yield a total points value, and the total points value can be correlated with the predictor scale to predict the quantitative probability of disease-specific death.

Also provided is a system. The system includes a processor; an input device; an output device; a storage device; a database wherein the database includes data collected from a plurality of patients previously diagnosed with and treated for breast cancer; software operable on the processor to: receive input from the input device, the input including one or more factors for determining death due to breast cancer; and correlate received input with the collected data from the plurality of patients previously diagnosed with and treated for breast cancer to determine a prognosis probability. In one embodiment, the determined prognosis includes a probabilities of recurrence of breast cancer and breast cancer survival. In another embodiment, the software further includes a Cox proportional hazards regression model for correlating input data with the collected data from the plurality of patients previously diagnosed with and treated for breast cancer. In yet another embodiment, the software further includes a neural network model for correlating input data with the collected data from the plurality of patients previously diagnosed with and treated for breast cancer. In a further embodiment, the neural network model is a non-linear, feed-forward system of layered neurons which back-propagate prediction errors. Also provided is a system wherein the software further includes a recursive partitioning model for correlating input data with the collected data from the plurality of patients previously diagnosed with and treated for breast cancer. In one embodiment, the software further includes vector machine technology for correlating input data with the collected data from the plurality of patients previously diagnosed with and treated for breast cancer. In a further embodiment, the system further includes a network connection, e.g., the network is the internet. In one embodiment, the database is a relational database management system. The system of the invention may include an output device that is a video display or a printer. The system may be a personal computer or a handheld computing device, e.g., a handheld computing device which includes PalmOS. The database of the system may be accessible via the network. The system may accept input and provide output over the internet, for instance, the input is received and the output is provided in a markup language such as HTML. The one or more factors include: tumor size; tumor grade; lymphovascular invasion; and/or tumor tissue staining.

Also provides is a machine-readable medium having instructions thereon for causing a suitably configured information-handling system to perform the methods of the invention.

Further provided is a system for predicting the recurrence of breast cancer. The system includes a data structure for storing historic breast cancer data, the structure contained in a memory and comprising a plurality of factors each corresponding to a characteristic of breast cancer; and a processing device including program means for correlating the plurality of factors corresponding to characteristics of breast cancer with factor data collected from a patient treated for breast cancer, wherein the correlating results in a probability of death due to breast cancer in the treated patient which is output by the processing device. The plurality of factors may include: tumor size; tumor grade; lymphovascular invasion; and/or tumor tissue staining.

In one embodiment, the invention provides a method for operating an information-processing device. The method includes maintaining a database of historic data wherein the historic data includes a plurality of scored factors corresponding to a plurality of previously treated patients; collecting scores from a current patient for the plurality of factors; and correlating the scores collected from the current patient with the historic data to determine a probability of death due to breast cancer.

### BRIEF DESCRIPTION OF THE FIGURES

FIG. 1. Cumulative incidence of death by NPI group. One woman with NPI=3, who was censored at 10 years, was excluded from the plot. Numbers at top indicate number of women at risk.

FIG. 2. Model calibration. The horizontal axis is the model's prediction of the probability of breast-cancer-specific death. The vertical axis is the actual breast-cancer-specific probability of death using the cumulative incidence method. The jackknife predicted probability was obtained for each patient, and patients were grouped into quartiles by this probability. Error bars represent 95% confidence intervals.

FIG. 3. Distribution of model predictions within each NPI risk group. One woman with NPI=III, who had a model predicted probability of 0.01, was excluded from the plot. Note the heterogeneity within NPI groups.

FIG. 4. Nomogram for 15-year breast-cancer-specific death. DSD=disease-specific death.

FIG. 5. Distribution of model predictions within tumor size categories (≦1 cm versus>1 cm) for women with nodes negative by IHC and H&E. Note the heterogeneity of prognoses within women with tumor size >1 cm.

FIG. 6. A block diagram of a system according to an embodiment of the invention.

FIG. 7. A schematic diagram of a system according to an embodiment of the invention.

FIG. 8. A block diagram of a computer readable medium with instructions thereon according to an embodiment of the invention.

### DETAILED DESCRIPTION OF THE INVENTION

The present invention provides methods, apparatus and nomograms to predict disease-specific death using factors available post-surgery to aid patients considering adjuvant therapy to treat breast cancer. In one embodiment, a nomogram predicts the probability of disease-specific death after mastectomy and/or axillary lymph node dissection using factors to assist the physician and patient in deciding whether or not the patient may benefit from adjuvant treatment protocols.

One embodiment of the invention is directed to a post-operative method for predicting the probability of disease-specific death in a breast cancer patient who has undergone a mastectomy and/or axillary lymph node dissection. The method comprises correlating one or more factors determined for each of a plurality of persons previously diagnosed with breast cancer prior to adjuvant therapy with the incidence of disease-specific death for each person of the plurality to generate a functional representation of the correlation. In alternative embodiments, one or more subgroups of any one or more of the following factors may be excluded. The factors comprise tumor size, tumor grade, lymphovascular invasion or staining, e.g., H&E staining, of tumor tissue, wherein said plurality of persons comprises females having undergone mastectomy and/or axillary lymph node dissection; and matching an identical set of factors determined from the patient to the functional representation to predict the probability of disease-specific death for the patient.

In one embodiment, the correlating includes accessing a memory storing the selected set of factors. In another embodiment, the correlating includes generating the functional representation and displaying the functional representation on a display. In one embodiment, the displaying includes transmitting the functional representation from a source. In one embodiment, the correlating is executed by a processor or a virtual computer program. In another embodiment, the correlating includes determining the selected set of factors. In one embodiment, determining includes accessing a memory storing the set of factors from the patient. In another embodiment, the method further comprises transmitting the quantitative probability of death from breast cancer. In yet another embodiment, the method further comprises displaying the functional representation on a display. In yet another embodiment, the method further comprises inputting the identical set of factors for the patient within an input device. In another embodiment, the method further comprises storing any of the set of factors to a memory or to a database.

In one embodiment, the nomogram is generated with a Cox proportional hazards regression model (Cox, 1972, the disclosure of which is specifically incorporated by reference herein). This method predicts survival-type outcomes using multiple predictor variables. The Cox proportional hazards regression method estimates the probability of reaching a certain end point, such as disease recurrence, over time. In another embodiment, the nomogram may be generated with a neural network model (Rumelhart et al., 1986, the disclosure of which is specifically incorporated by reference herein). This is a non-linear, feed-forward system of layered neurons which backpropagate prediction errors. In another embodiment, the nomogram may be generated with a recursive partitioning model (Breiman et al., 1984, the disclosure of which is specifically incorporated by reference herein). In yet another embodiment, the nomogram is generated with support vector machine technology (Cristianni et al., 2000; Hastie, 2001). In a further embodiment, e.g., for hormone refractory patients, an accelerated failure time model may be employed (Harrell, 2001). Other models known to those skilled in the art may alternatively be used. In one embodiment, the invention includes the use of software that implements Cox regression models or support vector machines to predict recurrence, disease-specific survival, disease-free survival and/or overall survival.

The nomogram may comprise an apparatus for predicting probability of disease-specific death in a patient with breast cancer following a mastectomy and/or axillary lymph node dissection and in the absence of adjuvant therapy. The apparatus comprises a correlation of one or more factors determined for each of a plurality of persons previously diagnosed with breast cancer and not having been treated adjuvant therapy with the incidence of disease-specific death for each person of the plurality of persons, the factors include tumor size, tumor grade, lymphovascular invasion or tumor tissue staining, and a means for matching an identical set of factors determined from the patient having breast cancer to the correlation to predict the probability of disease-specific death in the patient following mastectomy and/or axillary lymph node dissection and in the absence of adjuvant therapy.

The nomogram or functional representation may assume any form, such as a computer program, e.g., in a hand-held device, world-wide-web page, e.g., written in FLASH, or a card, such as a laminated card. Any other suitable representation, picture, depiction or exemplification may be used. The nomogram may comprise a graphic representation and/or may be stored in a database or memory, e.g., a random access memory, read-only memory, disk, virtual memory or processor.

The apparatus comprising a nomogram may further comprise a storage mechanism, wherein the storage mechanism stores the nomogram; an input device that inputs the identical set of factors determined from a patient into the apparatus; and a display mechanism, wherein the display mechanism displays the quantitative probability of disease-specific death in a patient with breast cancer, e.g., in a patient having undergone a mastectomy and in the absence of adjuvant therapy. The storage mechanism may be random access memory, read-only memory, a disk, virtual memory, a database, and a processor. The input device may be a keypad, a keyboard, stored data, a touch screen, a voice activated system, a downloadable program, downloadable data, a digital interface, a hand-held device, or an infra-red signal device. The display mechanism may be a computer monitor, a cathode ray tub (CRT), a digital screen, a light-emitting diode (LED), a liquid crystal display (LCD), an X-ray, a compressed digitized image, a video image, or a hand-held device. The apparatus may further comprise a display that displays the quantitative probability of disease-specific death in a patient with breast cancer, e.g., the display is separated from the processor such that the display receives the quantitative probability of disease-specific death in a patient with breast cancer, e.g., in a patient having undergone a mastectomy and in the absence of adjuvant therapy. The apparatus may further comprise a database, wherein the database stores the correlation of factors and is accessible by the processor. The apparatus may further comprise an input device that inputs the identical set of factors determined from the patient with breast cancer into the apparatus. The input device stores the identical set of factors in a storage mechanism that is accessible by the processor. The apparatus may further comprise a transmission medium for transmitting the selected set of factors. The transmission medium is coupled to the processor and the correlation of factors. The apparatus may further comprise a transmission medium for transmitting the identical set of factors determined from the patient with breast cancer, preferably the transmission medium is coupled to the processor and the correlation of factors. The processor may be a multi-purpose or a dedicated processor. The processor includes an object oriented program having libraries, said libraries storing said correlation of factors.

In one embodiment, the nomogram comprises a graphic representation of a probability that a patient with breast cancer will die of that disease following mastectomy and/or axillary lymph node dissection without adjuvant therapy. The nomogram comprises a substrate or solid support, and a set of indicia on the substrate or solid support, the indicia including one or more of a line for tumor size, tumor grade, lymphovascular invasion or staining of tumor tissue, a points line, a total points line and a predictor line, wherein the line for tumor size, tumor grade, lymphovascular invasion or staining of tumor tissue, each have values on a scale which can be correlated with values on a scale on the points line. The total points line has values on a scale which may be correlated with values on a scale on the predictor line, such that the value of each of the points correlating with the indicia can be added together to yield a total points value, and the total points value correlated with the predictor line to predict the probability of disease-specific death. The solid support may assume any appropriate form such as, for example, a laminated card. Any other suitable representation, picture, depiction or exemplification may be used.

In addition to assisting the patient and physician in selecting an appropriate course of therapy, the nomograms of the present invention are also useful in clinical trials to identify patients appropriate for a trial, to quantify the expected benefit relative to baseline risk, to verify the effectiveness of randomization, to reduce the sample size requirements, and to facilitate comparisons across studies.

The invention will be further described by the following non-limiting example.

### EXAMPLE I

Patients and Methods

519 consecutive women were treated with mastectomy and axillary lymph node dissection who met the following inclusion criteria: confirmation of invasive mammary carcinoma, no neoadjuvant or adjuvant systemic therapy, no prior history of cancer, and negative lymph nodes by routine pathologic analysis. Paraffin blocks were available for 368 of these 519 eligible cases. Enhanced pathologic analysis of the axillary lymph nodes was performed by sectioning tissue blocks at 2 deeper levels, 50 μm apart, and staining with hematoxylin and eosin (H&E) and immunohistochemistry (IHC) (AEl: AE3, Ventana Med Syst, Inc., Tucson, Ariz.). For the endpoint of disease-specific death, the following variables were selected as they are widely available and potentially prognostic: age, multifocality, tumor size, tumor grade, lymphovascular invasion, and enhanced staining. Patients with one or more missing values were excluded (multifocality, N=1; tumor size, N=2; tumor grade, N=17), leaving 348 complete patient records. Cause of death was recorded for patients who died.

Disease-specific death was estimated using the competing-risk method because nearly half of the deaths were due to other causes (Gray, 1988). A model was constructed based on analysis using the conditional cumulative incidence method (Fine et al., 1996). This model was the basis for a computerized prediction tool.

Model validation comprised two steps. First, discrimination was quantified with the concordance index (Harrell et al., 1982). Similar to the area under the receiver operating characteristic curve, but appropriate for censored data, the concordance index provides the probability that, in a randomly selected pair of patients in which one patient dies of disease before the other, the patient who died first had the worse predicted outcome from the model. Note that the second patient need not die of disease; she merely needs to survive longer than the first. Thus, the concordance index represents the fraction of these pairs of patients in whom the prediction model correctly identified the patient with the shorter survival time. Tossing a coin would be expected to achieve 50%.

In the second step, calibration was assessed. This was done by grouping patients with respect to their jackknife-calculated model predicted probabilities and then comparing the mean of the group with the observed cumulative incidence estimate of disease-specific death. All analyses were performed using S-Plus 2000 Professional software with the cmprsk, Design and Hmisc libraries added (Harrell, 2001).

The present prediction model was compared with that of the NPI as follows. First, jackknife predictions from the model were obtained for each woman by leaving her out of the dataset and refitting the model to the remaining women, and then obtaining her probability of death within 15 years. This leave-one-out analysis was performed for each woman. These predictions were compared with NPI by the concordance index. NPI was calculated as described by the Swedish Breast Cancer Cooperative Group (Group SBCC, 1996). Specifically, NPI was calculated as follows:
NPI=0.2×size[in cm]+nodal stage[1, 2, or 3]+grade[1, 2, or 3].

Nodal stage was assigned as follows: for a woman with no positive nodes, stage 1, stage 2 if she had 1-3 positive nodes, and stage 3 if more than 3 positive nodes by H&E. Survival predictions were obtained by grouping the patients as good, moderate, or poor risk when the NPI was ≦3.4, between 3.4 and 5.4, or >5.4, respectively (D'Eredita et al., 2001).

Results

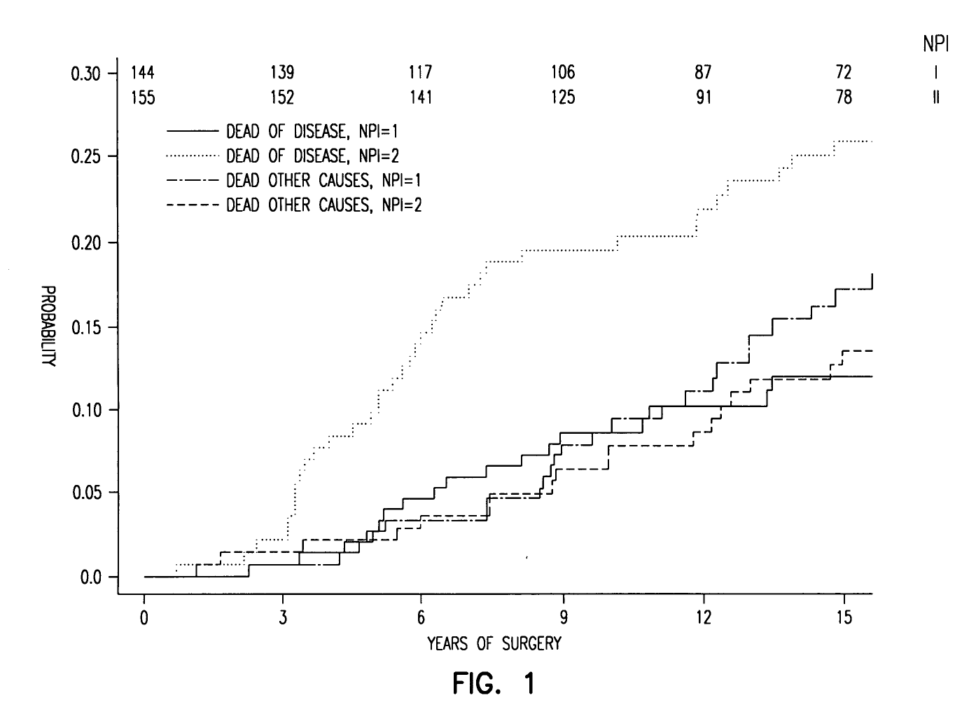

Descriptive statistics for this cohort appear in Table 1. At last follow-up, 73 patients had died of their disease, and 67 had died of other causes. Disease-specific death by NPI stage grouping is shown in FIG. 1.

TABLE 1
Descriptive Statistics for Breast Cancer Cohort

| Patient Characteristic | No. | % |
|---|---|---|
| Multifocality | | |
| No | 338 | 92.0 |
| Yes | 29 | 7.9 |
| NA | 1 | 0.2 |
| Grade | | |
| I | 58 | 16 |
| II | 134 | 36 |
| III | 110 | 30 |
| Lobular | 49 | 13 |
| NA | 17 | 5 |
| Lymphovascular Invasion | | |
| No | 318 | 86 |
| Yes | 50 | 14 |
| Staining | | |
| Pos IHC | 50 | 14 |
| Pos IHC and H + E | 33 | 9 |
| Neg IHC and H + E | 285 | 77 |
| Age | | |
| Minimum | | 24 |
| 1^{st} Quartile | | 47 |
| Median | | 52 |
| Mean | | 56 |
| 3^{rd} Quartile | | 64 |
| Maximum | | 83 |
| Size | | |
| Minimum | | 0.01 |
| 1^{st} Quartile | | 1.30 |
| Median | | 1.80 |
| Mean | | 1.90 |
| 3^{rd} Quartile | | 2.50 |
| Maximum | | 9.00 |
| NA | | 2.00 |

*NA = Not Available

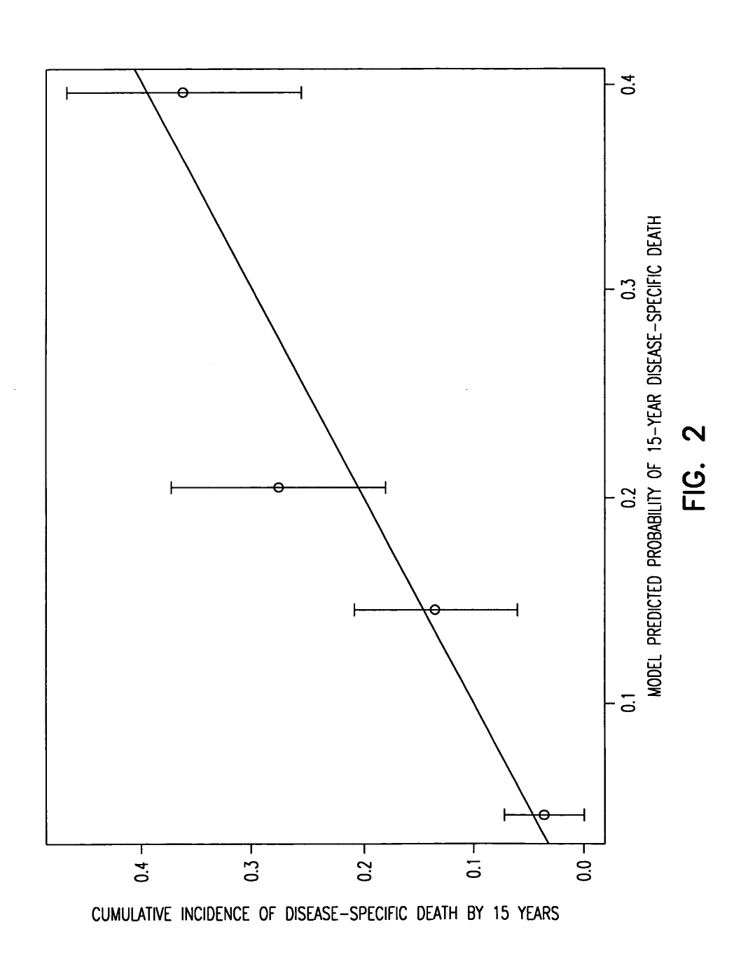

From the conditional cumulative incidence model, tumor size (P=0.006), tumor grade II vs I (P=0.010), tumor grade III vs I (P=0.012), tumor type “lobular” vs grade I (P=0.002), lymphovascular invasion (P=0.008), and H&E staining of the lymph nodes (P=0.005) were associated with disease-specific death, while age (P=0.270), multifocality (P=0.440), and IHC staining of the lymph nodes (P=0.800) were not. The concordance index for this model using jackknife predicted probabilities was 0.69. FIG. 2 illustrates the calibration of the model. For no quartile was the actual probability of disease specific death significantly different from the predicted probability.

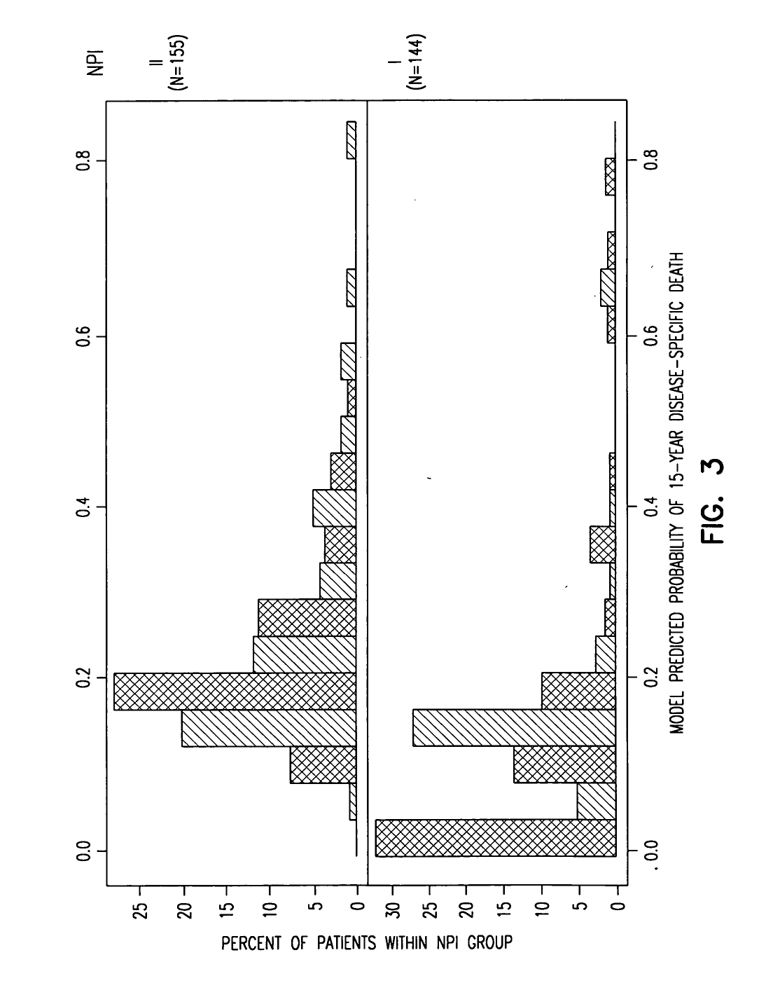

Finally, these predictions were compared with those obtained by using the NPI stage groupings. Individual NPI and model predictions were compared for their ability to rank the patients (e.g., concordance index) using the subset of patients applicable to both our model and the NPI (i.e., ductal carcinoma). To correct for overfit, model predictions were calculated on a leave-one-out basis, so that each patient was not included in the model that produced her prediction. Model discrimination was superior to that of NPI stage grouping (concordance index 0.70 vs 0.61, P=0.003). This improvement difference is difficult to appreciate clinically, and so, in FIG. 3 the discrepancies between the two prediction methods are illustrated. Within each NPI stage is a histogram of model-predicted probabilities. Note the heterogeneity of model predictions within the NPI I and II stages.

Note, also, that the NPI uses nodal stage based on H&E analysis. Some of the women, when their nodes were re-read, turned out to have had positive nodes despite those nodes having originally been declared negative, decades ago. The NPI scores were recomputed using the re-read H&E results. Employing the new nodal stage improved the performance of the NPI groupings (concordance index of 0.64 vs 0.61), but not to the level of the present prediction model (concordance index=0.69, P=0.014).

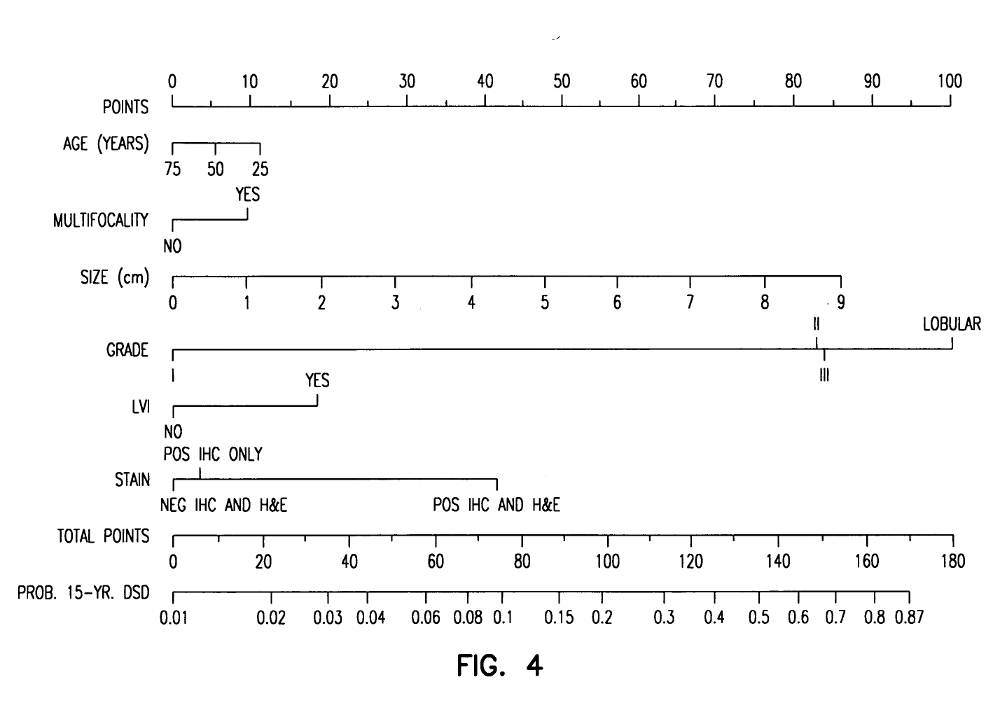

When a handheld or desktop computer is not practical, FIG. 4 provides the prediction tool as a nomogram. This is a graphic representation of the regression model, allowing the user to compute the patient's predicted probability of death from breast cancer within 15 years.

Discussion

The prognosis for a woman with negative nodes post-mastectomy for breast cancer is of critical importance. Among women considering adjuvant therapy, 91% would like to know their prognosis without adjuvant therapy (Lonn et al., 2001). However, when asked to recall such information after initiation of therapy, only 39% claim to have been told a quantitative estimate of their prognosis, and only 31% say they were provided quantitative estimates both with and without adjuvant therapy (Ravdin et al., 1998). Thus, for simply informing and counseling the patient, prognosis in this setting is critical and is, apparently, not being communicated well.

The decision regarding adjuvant therapy is exceedingly difficult. It is clear that adjuvant therapy is unpleasant and inconvenient (Duric et al., 2001), and provides modest benefits (Simes et al., 2001). Whether or not to have it is a legitimate decision (Duric et al., 2001) involving tradeoffs (Gelber et al., 1998). While the NCI clinical alert has suggested that all node-negative women should receive adjuvant therapy (Hillner et al., 1991), refinement of the determination of risk posed by the cancer is needed (Hillner et al., 1991; Hillner et al., 1991).

In this study, enhanced pathologic analysis of a cohort of women treated for breast cancer with mastectomy, but who did not receive adjuvant therapy, was performed. It was found that, with enhanced pathologic assessment, 9% of them actually had H&E positive lymph nodes. This enhanced assessment was associated with disease-specific death in multivariable analysis (P=0.005). Using this assessment and other variables, a model to predict a continuous probability of disease-specific death was developed, and it produced a tool that appears to predict death more accurately than previous models. When compared with the NPI, the model predicted more accurately when measured by the concordance index (P<0.02).

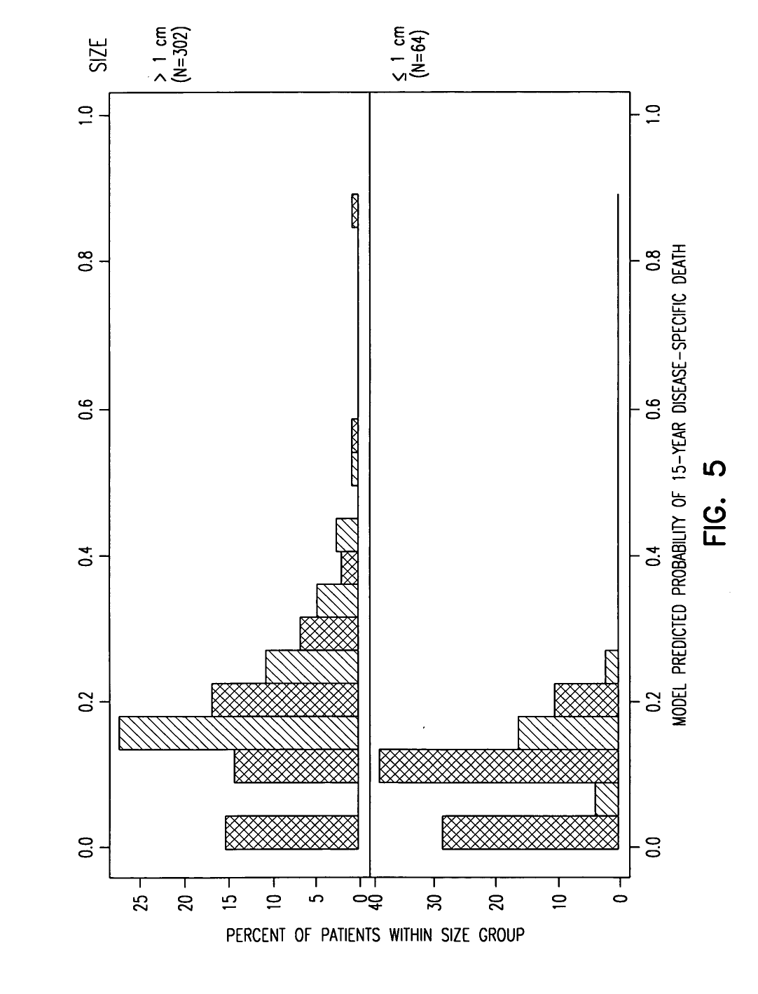

The model suggests, as others have found (Rosen et al., 1991; Carter et al., 1989), that tumor size is an important prognostic variable. However, it is clear that a cutoff point in tumor size is problematic. Simply put, the bigger the tumor, the worse the prognosis, if all other prognostic factors are held constant. Use of a heuristic, such as a 1 cm cutoff (Rosen et al., 1989), will result in inferior predictive accuracy for counseling and managing a patient. Furthermore, categorizing tumor size loses very valuable information. Instead, when counseling or deciding upon adjuvant therapy, all of a woman's prognostic factors should be considered in an optimal fashion to produce the most accurate prediction possible. A computerized version of the prediction model would provide an important step in this direction and might be the most accurate prediction method currently available. FIG. 5 illustrates the model predictions in node negative women relative to use of a size >1 cm heuristic. Note the heterogeneity of women in the “high risk” category. Note also that some women in the ≦1 cm category have probability of death >20 %. These women should not be counseled or managed in a uniform fashion. However, if one were to use the lcm cutoff rule, they would be. In contrast, our model clearly separates these women based on their prognoses. The model, with a concordance index of 0.69, predicts better than the categorization of women as tumor size ≦ or >1 cm (Rosen et al., 1989) (concordance index=0.53), or tumor size <2 cm vs 2-4.9 cm vs ≧5 cm (Carter et al., 1989) (concordance index=0.60).

The present work is similar in spirit, though more limited in scope, to that of Ravdin et al. (2001) and Loprinzi and Thome (2001). Their approaches go well beyond the present approached by examining the effect of adjuvant therapy on subsequent risk of recurrence and death. The present model lacks this prediction but does take more information into account when making a prediction of death in the absence of adjuvant therapy. For example, in the present model, tumor grade and method of staining, which are not present in the other models, were each statistically significant predictors of disease-specific death. The other models predict survival to 10 years, while the present model predicts to 15 years, a difference that hampers direct comparison of the models.

In addition to its use simply for patient counseling, the model can be useful in a physician's decision regarding adjuvant therapy. The tool provides a predicted probability of death in the absence of adjuvant therapy. Presumably, a patient with a very low baseline risk might wish to avoid the toxicity associated with adjuvant therapy. Communicating this risk should be helpful in any discussion regarding this difficult decision. Thus, the prediction tool can be an effective decision aid (Whelan et al., 2003). This prediction tool might also be useful as a benchmark for judging the predictive ability of any new technology, such as gene expression analysis. It is hoped that, in the future, such novel technologies will be widely available and able to predict with better accuracy than that achieved with the present model (i.e., a concordance index >0.69).

In conclusion, was a tool for predicting disease specific death with 15-years fro women with breast cancer treated with mastectomy alone, was developed and internally validated. The tool appears to improve upon the existing ability to predict with the NPI.

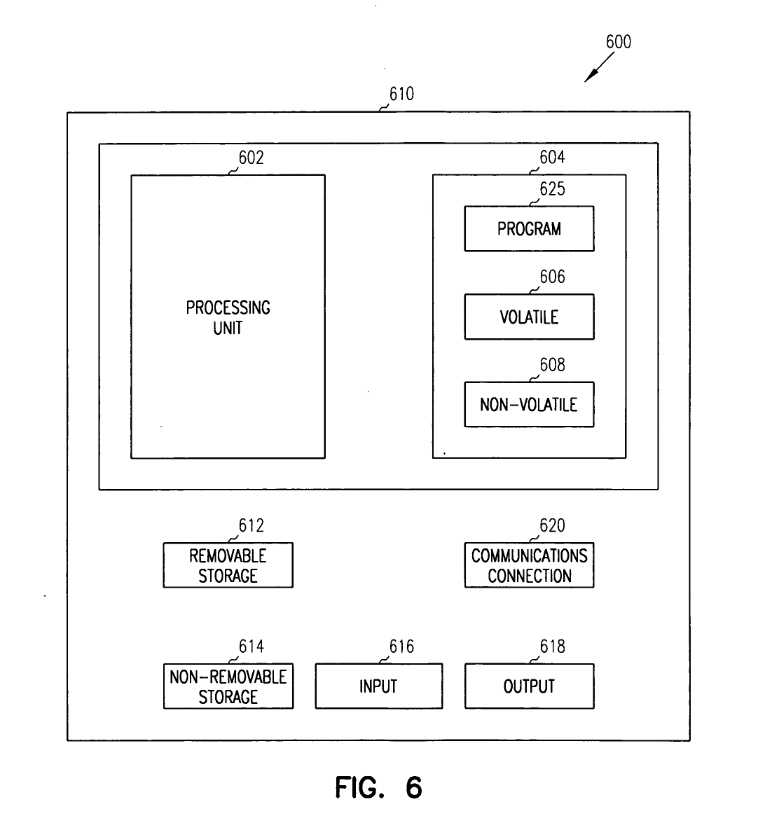

A block diagram of a computer system that executes programming for predicting a prognosis probability is shown in FIG. 6. A general computing device in the form of a computer 610, may include a processing unit 602, memory 604, removable storage 612, and non-removable storage 614. Memory 604 may include volatile memory 606 and non-volatile memory 608. Computer 610 may include— or have access to a computing environment that comprises—a variety of computer-readable media, such as volatile memory 606 and non-volatile memory 608, removable storage 612 (and 800 as shown in FIG. 5) and non-removable storage 614. Computer storage comprises RAM, ROM, EPROM & EEPROM, flash memory or other memory technologies, CD ROM, Digital Versatile Disks (DVD) or other optical disk storage, magnetic cassettes, magnetic tape, magnetic disk storage or other magnetic storage devices, or any other medium capable of storing computer-readable instructions. Computer 610 may include or have access to a computing environment that comprises input 616, output 618, and a communication connection 620. Input 616 may include one or several devices such as a keyboard, mouse, touch screen, and stylus. Output 618 may include one or several devices such as a video display, a printer, an audio output device, a touch stimulation output device, or a screen reading output device. The computer may operate in a networked environment using a communication connection 620 to connect to one or more remote computers. The remote computer may include a personal computer, server, router, network PC, a peer device or other common network node, or the like. The communication connection 620 may include a Local Area Network (LAN), a Wide Area Network (WAN) or other networks. The communication connection 620 may be over a wired network, wireless radio frequency network, or an infrared network. Further, in some embodiments, the network may be a combination of several connection technologies including wired, RF, and/or infrared.

Computer-readable instructions stored on a computer-readable medium are executable by the processing unit 602 of the computer 610. A hard drive, CD-ROM, and RAM are some examples of articles including a computer-readable medium. The computer-readable instructions allow computer system 600 to provide generic access controls in a computer network system having multiple users and servers, wherein communication between the computers includes utilizing TCP/IP, COM, DCOM, XML, Simple Object Access Protocol (SOAP), and Web Services Description Language (WSDL), and other related connection communication protocols and technologies that will be readily apparent to one of skill in the relevant art.

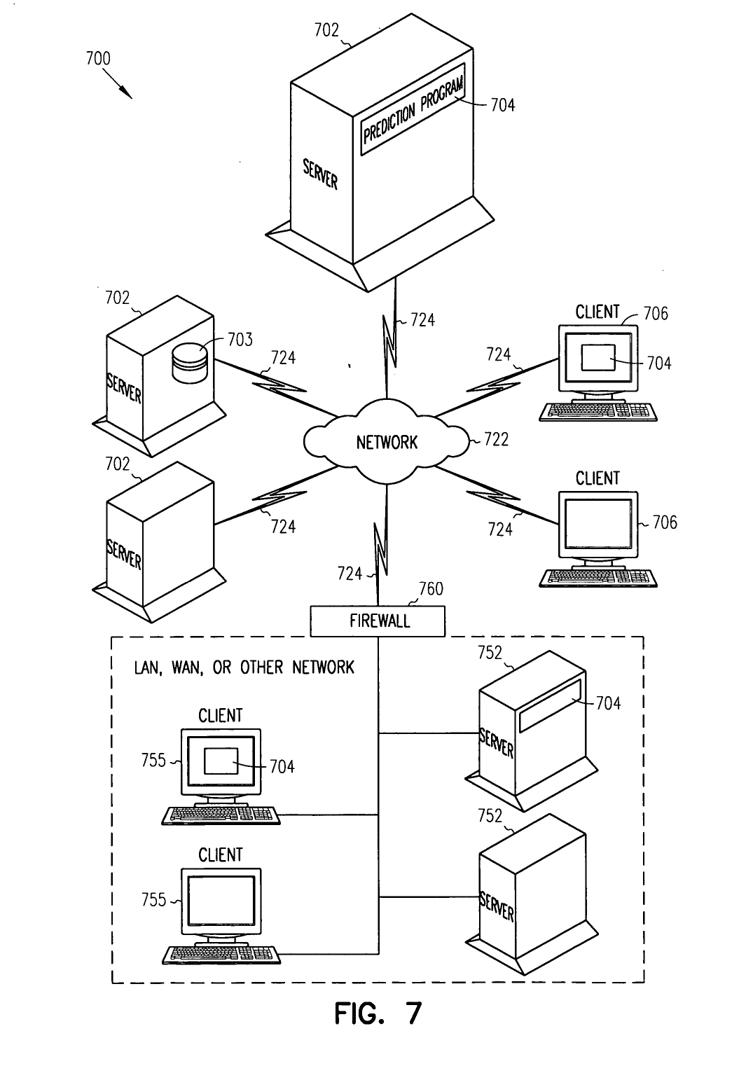

FIG. 7 shows an exemplary embodiment of an information processing system 700 that provides for transfer of data between multiple devices. This embodiment of system 700 comprises multiple servers 702, client work stations 706, the servers 702 and client workstations 706 operatively connected via communication lines 724 to a network 722. In one embodiment, network 722 includes the Internet, or other type of public or private network that allows data transfer. Communication lines 724 may be any type of communication medium, such as telephone lines, cable, optical fiber, wireless, or any other communication medium that allows data transfer between devices coupled to the network.

In some embodiments, one or more of the servers 702 hold a prediction program 704, which is available for download to the other servers 702 and workstation clients 706 connected 724 to the network 722.

In some other embodiments, prediction program 704 is executable on a server 702 wherein the prediction program executes in response to stimulation received from a client 704 using a Hyper-Text Transfer Protocol (HTTP or HTTPS). In one such embodiment, prediction program 704 accepts input from a client, executes, and outputs a prognosis prediction in a markup language such as Hyper-Text Markup Language (HTML) or eXtensible Markup Language (XML).

In some embodiments, system 700 may be implemented with servers 702 utilizing one of many available operating systems. Servers 702 may also include, for example, machine variants such as personal computers, handheld personal digital assistants, RISC processor computers, MIP single and multiprocessor class computers, and other personal, workgroup, and enterprise class servers. Further, servers 702 may also be implemented with relational database management systems 703 and application servers. Other servers 702 may be file servers.

Client workstations 702 within embodiments of system 700, may include personal computers, computer terminals, handheld devices, and multifunction mobile phones. Client workstations 702 include software thereon for performing operations in accordance with stimulation received from a user and signals received from other computing devices on the network 722. Further, a client workstation 702 may include a web browser for displaying web pages.

The network 722 within some embodiments of a system 700 may include a Local Area Network (LAN), Wide Area Network (WAN), or other similar network 745 connected 724 network 722. Network 722 may itself be a LAN, WAN, the Internet, or other large scale regional, national, or global network or a combination of several types of networks. Some embodiments of system 700 include a LAN, WAN, or other similar network 745 that utilizes one or more servers 752 and clients 755 behind a firewall 760 within the LAN, WAN, or other similar network 745.

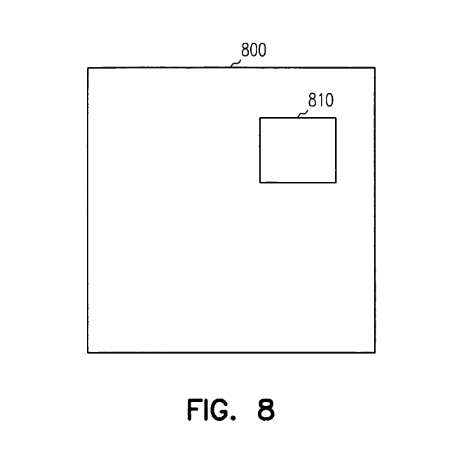

FIG. 8 shows an exemplary embodiment of a machine-readable medium 800 with operable instructions 810 thereon for performing the methods described herein on an appropriately configured information processing device. Such devices include in various embodiments personal computers including desktop, laptop, and tablet computers. Some further embodiments include handheld devices utilizing Palm/OS or Windows CE.

References

Carter et al., Cancer. 1989;63(1):181-187.
D'Eredita et al., Eur J Cancer. 2001;37:591-596.
Duric et al., Lancer Oncol. November 2001;2(11):691-697.
Fine et al., JASA. 1999;94(446):496-509.
Gelber et al., Recent Results Cancer Res. 1998;152:373-389.
Gray et al., Annals of Statistics. 1988;16:1141-1154.
Group SBCC. Randomized Trial of Two Versus Five Years of Adjuvant Tamoxifen Postmenopausal Early Stage Breast Cancer. Journal of the National Cancer Institute. 1996;88(21):1543-1549.
Harrell et al., In: Regressions Modeling Strategies With Applications to Linear Models, Logistic Regression, and Survival Analysis. New York: Springer-Verlag; 2001.
Harrell et al., JAMA. May 1982;247(18):2543-2546.
Hillner et al., Breast Cancer Res Treat. 1992;23(1-2):17-27.
Hillner et al., N Engl J Med. Jan. 17 1991;324(3):160-168.
Kattan et al., J Clin Oncol. 2000:18:3352-3359.
Kattan et al., J Clin Oncol. February 2002;20(3):791-796.
Kattan et al., State Med. 2003;In Press.
Lobb et al., Health Expect. March 2001;4(1):48-57.
Loprinzi et al., Journal of Clinical Oncology. Feb. 15, 2001; 19(4):972-979.
Mincey et al., Oncologist. 2002;7(3):246-250.
Ravdin et al., J Clin Oncol. Febuary 1998;16(2):515-521.
Ravdin et al., J Clin Oncol. Feb. 15, 2001;19(4):980-991.
Rosen et al., Journal of Clinical Oncology. March 1989;7(3):355-366.
Rosen et al., Journal of Clinical Oncology. September 1991;9(9):1650-1661.
Simes et al., J Natl Cancer Inst Monogr. 2001(30):146-152.
Whelan et al., Journal of the National Cancer Institute. 2003;95(8):581-587.
Breiman et al., Classification and Regression Trees. Monterey, Calif., Wadsworth and Brooks/Cole (1984).
Cristianni et al., An Introduction to Support Vector Machines, Cambridge University Press (2000).
Hastie, The Elements of Statistical Learning, Springer (2001).
Rumelhart et al., (eds), Parallel Distributed Processing: Exploration in the Microstructure of Cognition Volume 1, Foundations Cambridge, Mass., The MIT Press (1986).
Cox, J. Royal Statistical Society, B34, 187-220 (1972).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method to determine the risk of disease-specific death in a breast cancer patient, comprising:
a) detecting or determining one or more factors comprising tumor size, tumor grade, lymphovascular invasion and/or tumor tissue staining, in a breast cancer patient prior to adjuvant therapy; and
b) correlating one or more of the factors to the risk of disease-specific death of the patient in the absence of adjuvant therapy.

2. A method to determine the prognosis of a breast cancer patient in the absence of adjuvant therapy, comprising:
a) inputting test information to a data input means, wherein the information comprises one or more factors comprising tumor size, tumor grade, lymphovascular invasion and/or tumor tissue staining in a breast cancer patient prior to adjuvant therapy;
b) executing a software for analysis of the test information; and
c) analyzing the test information so as to provide the prognosis of the patient in the absence of adjuvant therapy.

3. A method for predicting a probability of disease-specific death in a breast cancer patient, comprising:
a) correlating one or more factors for the patient to a functional representation of one or more factors determined for each of a plurality of persons previously diagnosed with breast cancer and not having been treated with adjuvant therapy, so as to yield a value for total points for the patient, which factors for each of a plurality of persons is correlated with the probability of disease-specific death for each person in the plurality of persons, wherein the one or more factors comprises tumor size, tumor grade, lymphovascular invasion or tumor tissue staining, wherein the functional representation comprises a scale for one or more of tumor size, tumor grade, lymphovascular invasion and/or tumor tissue staining, a total points scale, and a predictor scale, wherein the scales for tumor size, tumor grade, lymphovascular invasion and/or tumor tissue staining each have values on the scales which can be correlated with values on the points scale, and wherein the total points scale has values which may be correlated with values on the predictor scale; and
b) correlating the value on the total points scale for the patient with a value on the predictor scale to predict the probability of disease-specific death in the patient in the. absence of adjuvant therapy.

4. The method of claim 3 wherein the functional representation is a nomogram.

5. The method of claim 4 wherein the nomogram is generated with a Cox proportional hazards regression model.

6. The method of claim 1 or 3 wherein the correlating is conducted by a computer.

7. An apparatus, comprising:
a data input means, for input of test information comprising one or more of tumor size, tumor grade, lymphovascular invasion or tumor tissue staining in a breast cancer patient prior to adjuvant therapy;
a processor, executing a software for analysis of tumor size, tumor grade, lymphovascular invasion or tumor tissue staining;
wherein the software analyzes the tumor size, tumor grade, lymphovascular invasion or tumor tissue staining and provides the risk of disease-specific death in the patient in the absence of adjuvant therapy.

8. The apparatus of claim 7 wherein the test information is input manually using the data input means.

9. The apparatus of claim 7 wherein the software constructs a database of the test information.

10. A nomogram for the graphic representation of a quantitative probability of disease-specific death in a patient with breast cancer, comprising: a plurality of scales and a solid support, the plurality of scales being disposed on the support and comprising a scale of one or more of tumor size, tumor grade, lymphovascular invasion and/or tumor tissue staining, a points scale, a total points scale and a predictor scale, wherein the one or more scales for tumor size, tumor grade, lymphovascular invasion and/or tumor tissue staining each has values on the scales, and wherein the scales for tumor size, tumor grade, lymphovascular invasion and/or tumor tissue staining are disposed on the solid support with respect to the points scale so that each of the values on tumor size, tumor grade, lymphovascular invasion and/or tumor tissue staining can be correlated with values on the points scale, wherein the total points scale has values on the total points scale, and wherein the total points scale is disposed on the solid support with respect to the predictor scale so that the values on the total points scale may be correlated with values on the predictor scale, such that the values on the points scale correlating with the tumor size, tumor grade, lymphovascular invasion and/or tumor tissue staining of the patient can be added together to yield a total points value, and the total points value can be correlated with the predictor scale to predict the quantitative probability of disease-specific death.

11. The nomogram of claim 10 wherein the solid support is a laminated card.

12. A system comprising:
a processor;
an input device;
an output device;
a storage device;
a database wherein the database includes data collected from a plurality of patients previously diagnosed with and treated for breast cancer;
software operable on the processor to:
receive input from the input device, the input including one or more factors for determining death due to breast cancer; and
correlate received input with the collected data from the plurality of patients previously diagnosed with and treated for breast cancer to determine a prognosis probability.

13. The system of claim 12 wherein the determined prognosis includes a probabilities of recurrence of breast cancer and breast cancer survival.

14. The system of claim 12 wherein the software further includes a Cox proportional hazards regression model for correlating input data with the collected data from the plurality of patients previously diagnosed with and treated for breast cancer.

15. The system of claim 12 wherein the software further includes a neural network model for correlating input data with the collected data from the plurality of patients previously diagnosed with and treated for breast cancer.

16. The system of claim 15 wherein the neural network model is a non-linear, feed-forward system of layered neurons which back-propagate prediction errors.

17. The system of claim 12 wherein the software further includes a recursive partitioning model for correlating input data with the collected data from the plurality of patients previously diagnosed with and treated for breast cancer.

18. The system of claim 12 wherein the software further includes vector machine technology for correlating input data with the collected data from the plurality of patients previously diagnosed with and treated for breast cancer.

19. The system of claim 12 further comprising a network connection.

20. The system of claim 19 wherein the network is the internet.

21. The system of claim 12 where the database is a relational database management system.

22. The system of claim 12 wherein the output device is a video display.

23. The system of claim 12 wherein the output device is a printer.

24. The system of claim 12 wherein the system is a personal computer.

25. The system of claim 12 wherein the system is a handheld computing device.

26. The system of claim 25 wherein the handheld computing device includes PalmOS.

27. The system of claim 19 wherein the database is accessible via the network.

28. The system of claim 20 wherein the system accepts input and provides output over the internet.

29. The system of claim 28 wherein the input is received and the output is provided in a markup language.

30. The system of claim 29 wherein the markup language is HTML.

31. The system of claim 12 wherein the one or more factors include:
tumor size;
tumor grade; and/or
lymphovascular invasion or tumor tissue staining.

32. A system for predicting the recurrence of breast cancer, the system comprising:
a data structure for storing historic breast cancer data, the structure contained in a memory and comprising a plurality of factors each corresponding to a characteristic of breast cancer; and
a processing device including program means for correlating the plurality of factors corresponding to characteristics of breast cancer with factor data collected from a patient treated for breast cancer, wherein the correlating results in a probability of death due to breast cancer in the treated patient which is output by the processing device.

33. The system of claim 32 wherein the plurality of factors include:
tumor size;
tumor grade; and/or
lymphovascular invasion or tumor tissue staining.

34. A method for operating an information-processing device comprising:
maintaining a database of historic data wherein the historic data includes a plurality of scored factors corresponding to a plurality of previously treated patients;
collecting scores from a current patient for the plurality of factors; and
correlating the scores collected from the current patient with the historic data to determine a probability of death due to breast cancer.
